(19) United States
(12) Patent Application Publication
(10) Pub. No.: US 2005/0065184 A1
(43) Pub. Date: Mar. 24, 2005

(54) Method of reducing the risk of oxidative stress

(75) Inventor: Gerald Wolf, Westborough, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: aaiPharma Inc.

(21) Appl. No.: 10/927,790

(22) Filed: Aug. 27, 2004

Related U.S. Application Data
(60) Provisional application No. 60/499,153, filed on Aug. 29, 2003.

(51) Int. Cl. A61K 31/454; A61K 31/401

(52) U.S. Cl. 514/323; 514/423

(57) ABSTRACT

A method for ascertaining whether a subject has oxidative stress; evaluating the level of oxidative stress in a subject; reducing the risk of an adverse event, especially an adverse cardiovascular event, resulting from oxidative stress; treating oxidative stress; and evaluating the efficacy of treatment with at least one pharmaceutical composition for reducing oxidative stress is provided.

### CROSS-REFERENCE

This application claims the benefit of priority from U.S. provisional application Ser. No. 60/499,153, filed Aug. 29, 2003 which is incorporated by reference in its entirety.

### BACKGROUND OF THE INVENTION

1. Field of the Invention

The invention is related to a method of reducing the risk of an oxidative stress-related event in a human subject. More particularly, the invention is related to treating oxidative stress in a subject to reduce the risk of a cardiovascular event in the subject.

2. Description of Related Art

The list of potential adverse cardiovascular events is substantial and includes a number of life-threatening conditions such as, for example, stroke, myocardial infarction, transient ischemic attacks, congestive heart failure, left ventricular hypertrophy, coronary artery disease, carotid artery disease, peripheral artery disease, and death. Many risk factors related to cardiovascular events are well known and include, for example, hypertension, smoking, diabetes, elevated cholesterol, obesity and metabolic syndrome. In particular, the effects of smoking and obesity on the cardiovascular system have been the focus of intense study in recent years. The marked increase in the incidence of overweight and obese persons is recognized as one of the most serious public health issues in the United States. It is estimated that currently greater than 60% of American adults are overweight and greater than 30% are obese, and such states are associated with a significantly increased mortality rate from atherosclerotic cardiovascular disease. Similarly, it is estimated that 20% of deaths from cardiovascular disease can be attributed to cigarette smoking in the United States. Bazzano L A et al. 2003. Among middle aged women, smoking has been reported to account for 50% of all cardiovascular events. Bermudez E A et al. 2002.

The relationship of the various risk factors for cardiovascular events and the actual onset of such an event has been the focus of the Framingham Heart Study. This study was initiated in a generally healthy population in 1948; subjects were studied over decades and the study has been updated to include the offspring of the original cohort. Due in large part to the Framingham Heart Study, it has been widely accepted that the relative risk of cardiovascular disease can be predicted in individuals by measuring certain factors in the patient. For example, total cholesterol, LDL cholesterol, and hypertension are factors that are now traditionally recognized as predictive of the risk of a future cardiovascular event and are now accepted surrogates for treatments intended to reduce that risk. Other factors now included in the Framingham Heart study are age, tobacco use, Body Mass Index (BMI), and diabetes.

More recently, it has been recognized that the existence of atherosclerosis is frequently accompanied by elevated biomarkers related to the identification of the process known as “oxidative stress.” This process can lead to actual vascular damage. Such identified biomarkers include, for example, measurable amounts of C-reactive protein, interleukin-6, fibrinogen, plasminogen activator inhibitor type 1 (PAI-1), and urinary isoprostanes. Ridker P M 2003, Pearson T A et al 2003, Dzau V J 2001, Keaney et al 2003, Nordt T K, 1999.

The biochemical mechanism of the relationship that obesity, smoking and the other commonly recognized risk factors have in the origin and the perpetuation of atherosclerosis has been an area of intense research, but many aspects of this relationship remain poorly understood. Atherosclerosis appears to originate from subclinical abnormalities with early manifestations such as, for example, diminished endothelium-dependent vasodilation or increased inner artery wall thickness. Over the last decade, there has been considerable interest in attempting to identify the role of oxidative stress in disease, particularly vascular disease. This interest has been driven by a wealth of data indicating that low density lipoprotein (LDL) oxidation is a prominent feature of atherosclerosis. More recently, studies have also suggested that oxidative stress biomarkers are associated with premature atherosclerosis, particularly in obese individuals, those that smoke, and those diagnosed as having diabetes and/or hypertension. (Keaney et al2003). Thus, enhanced oxidative stress, occurring either locally in the arterial wall or systemically, may contribute to the further development of atherosclerosis, atheroscleritis, atherothrombosis, or other cardiovascular diseases.

The mechanisms of vascular damage due to oxidative stress have been studied relative to specific risk factors. In particular, it has been hypothesized that oxidative modification of the lipid components of LDL may be at least one significant cause in the formation of atherosclerosis. LDL is deposited in the vascular wall early in the course of atherosclerotic lesion development, where it is subsequently oxidized. Evidence obtained from both in vitro and animal models of human atherosclerosis demonstrate that oxidized lipids derived from LDL contribute to many of the stages of atherosclerotic development. Pearson T A et al. 2003.

Measurement of the biomarkers for oxidative stress can provide a method of predicting disease risk, including a risk of an adverse cardiovascular event before traditional risk factors raise a level of concern. For example, measurement of urinary isoprostanes is one of the most accurate methods to quantify oxidative stress in humans. Keaney et al. 2003, Block et al. 2003.

Urinary isoprostanes are typically found in increased concentrations in subjects having hypercholesterolemia, diabetes mellitus, and hyperhomocysteinemia, those suffering from obesity, and in chronic heavy cigarette smokers. These observations suggest that certain populations known to be at risk for developing atherosclerosis are also under increased oxidative stress. Keaney et al. 2003. To date, however, the medical community has focused on the occurrence of actual cardiovascular events, diagnosis of certain disease states, or the presence of conventional risk factors to trigger the use of appropriate pharmaceutical agents. While animal and human epidemiologic studies carried out in the 1980s and 1990s suggested that antioxidants decrease atherosclerosis, prospective clinical trials of antioxidant supplementation using vitamin E and other agents have been disappointing because they failed to reduce cardiovascular events. Morrow et al. 2003.

Currently, treatment to prevent occurrence or recurrence of cardiovascular events is generally determined based upon the diagnosis of disease states by using the traditional surrogates for cardiovascular disease. Treatment includes use of pharmaceutical agents targeted for the disease state associated with the surrogate. For example, a common treatment for hypertension is an inhibitor of angiotensin converting enzyme, an ACE inhibitor, or an angiotensin receptor blocker. These classes of drugs are targeted to the renin-angiotensin system which plays a pivotal role in regulating blood pressure. Other classes of compounds, such as the statins, are administered for treatment of hyperlipidemia. Very recently, it has been suggested that statin compounds be used in the absence of hyperlipidemia. Wald et al. 2003, Wald et al. 2003; Law et al. 2003. In addition, numerous drugs have been used to reduce the occurrence of adverse cardiovascular events in diabetic patients, as well as patients with established cardiac disease such as heart failure and left ventricular hypertrophy. However, such drug regiments have not been predicated upon the levels of biomarkers of oxidative stress, nor were these levels used to guide the selection or regimen. Thus, risk factors of cardiovascular events, such as hypertension, hypercholesterolemia, diabetes, and previous cardiovascular events such as myocardial infarction or congestive heart failure, that have been shown to respond favorably to treatment with antihypertensive or anticholesterolemic drugs, can be distinguished from oxidative stress biomarkers. Therapy to lower the risk of cardiovascular events guided by measurement of the biomarkers of oxidative stress has not been recommended.

Accordingly, what is needed is therapy for treating oxidative stress before the consequences of oxidative stress progress to the stage where traditional risk factors or surrogates are present, atherosclerosis is clinically evident or a cardiovascular event has occurred. Such therapy can be based upon an elevated level of at least one of the patient's biomarkers for oxidative stress and can be initiated prior to the initiation of treatment based on traditional risk factors, surrogates, specific observable disease states and/or observable cardiovascular events.

The complete citations to the references cited above and further herein can be found at the end of this specification in the section entitled REFERENCES.

### SUMMARY OF THE INVENTION

In one aspect, the invention is directed to a method of treating a patient at risk of an adverse event as the result of having elevated oxidative stress, more particularly where the event at risk is a cardiovascular event. The method includes administering an effective amount of a pharmaceutical composition for reducing, or preventing an increase of the level of at least one biomarker for oxidative stress. The pharmaceutical composition is administered prior to the time indicated for the administration of compounds for treating cardiovascular events based upon traditional risk factors for adverse cardiovascular events.

In another aspect, the invention is directed to a method of lowering the risk of an adverse cardiovascular event in a patient having elevated oxidative stress comprising treating the patient to lower, or prevent an increase in the level of, at least one biomarker for oxidative stress. Treatment includes the administration of a pharmaceutical composition for lowering the oxidative stress prior to the time indicated for reducing adverse cardiovascular events based upon the traditional risk factors for such events.

In a further aspect, the invention is directed to a method of lowering the risk of an adverse cardiovascular event in a human subject by measuring in the subject the level of at least one biomarker for oxidative stress and treating those subjects having an elevated level of at least one of the biomarkers to lower, or prevent an increase in, the level of at least one of the biomarkers. Administration is conducted prior to an increase in the level of a traditional risk factor for a cardiovascular event.

In yet another aspect, the invention is directed to a method of treating a patient at risk of an adverse cardiovascular event. The method includes measuring the patient's risk of an adverse cardiovascular event by determining the patient's level of oxidative stress; administering to the patient an amount of a pharmaceutical composition for treating oxidative stress; measuring the level of oxidative stress during administration of the pharmaceutical composition and terminating the administration or modifying the amount administered of the pharmaceutical composition when it is concluded that the consequences of further administration outweigh a benefit of a lower risk. In yet another aspect, the invention is directed to a method for evaluating the level of oxidative stress in a subject. The method includes measuring the oxidative stress of a subject by testing for at least one biomarker for oxidative stress from said subject; assigning previously measured biomarker data into a number of windows, wherein each window has a weighted value; comparing the value of the tested biomarker to at least one window so as to obtain an oxidative stress score component; aggregating the oxidative stress score component to produce an oxidative stress score; and comparing the oxidative stress score to a previously determined threshold value.

In yet another aspect, the invention is directed to a method for treating a subject suspected of having oxidative stress. The method includes measuring the oxidative stress of a subject by testing for at least one biomarker for oxidative stress from said subject; determining whether the at least one tested biomarker is indicative of oxidative stress; and wherein when the at least one tested biomarker is indicative of oxidative stress, treating said subject to reduce or prevent an increase of the level of the least one biomarker for oxidative stress.

In yet another aspect, the invention is directed to a method for ascertaining whether a subject has oxidative stress. The method includes measuring the oxidative stress of a subject by testing for at least one biomarker for oxidative stress from said subject and determining whether the at least one tested biomarker is indicative of oxidative stress.

In yet another aspect, the invention is directed to a method for treating a subject suspected of having oxidative stress. The method includes measuring the oxidative stress of a subject by testing for at least one biomarker for oxidative stress from said subject; determining whether the at least one tested biomarker is indicative of oxidative stress; and wherein when the at least one tested biomarker is indicative of oxidative stress, treating said subject to reduce or prevent an increase of the level of the least one biomarker for oxidative stress.

In yet another aspect, the invention is directed to a method for evaluating the level of oxidative stress in a subject. The method includes measuring the oxidative stress of a subject by testing for at least one biomarker for oxidative stress from said subject; providing an arrangement of windows, wherein each window has a weighted value based on previously measured biomarker data; comparing the value of the tested biomarker to at least one window so as to obtain an oxidative stress score component; aggregating the oxidative stress score component to produce an oxidative stress score; and comparing the oxidative stress score to a previously determined threshold value.

In yet another aspect, the invention is directed to a method for determining the effectiveness of at one pharmaceutical composition for reducing the risk of an adverse cardiovascular event in a subject having elevated oxidative stress. The method includes: (a) measuring a first level of oxidative stress of a subject by testing for at least one biomarker for oxidative stress; (b) administering at least one pharmaceutical composition to said subject to reduce the level of at least one biomarker for oxidative stress; measuring a second level of oxidative stress of said subject by testing for the at least one biomarker for oxidative stress; and (d) comparing the values of the first and second levels of oxidative stress so as to determine whether the at least one pharmaceutical composition is effective in reducing the risk of an adverse cardiovascular event or it is concluded that the consequences of further administration outweigh a benefit of a lower risk.

### DETAILED DESCRIPTION OF THE INVENTION

Before describing the present invention in detail, it is to be understood that this invention is not limited to specific dosage forms, carriers, or the like, as such may vary. It is also to be understood that the terminology used herein is for the purpose of describing particular embodiments only, and is not intended to be limiting.

In describing the present invention, the following terminology will be used in accordance with the definitions set out below. As used herein, the singular forms a, an and “the” include plural referents unless the context clearly dictates otherwise. Thus, for example, reference to “an active agent” or “a pharmacologically active agent” includes a single active agent as well as two or more different active agents in combination. Similarly, reference to “a carrier” includes mixtures of two or more carriers as well as a single carrier, and the like.

“Oxidative stress” refers to a condition caused by the presence of free radicals or radical-generating agents in concentrations that overwhelm natural radical-blocking or radical-scavenging mechanisms. Sources of oxidative stress include, for example, exogenous factors such as, for example, cigarette smoke, and endogenous factors such as, for example, the oxidative burst from activated macrophages. Oxidative stress can cause oxidative damage to DNA, proteins, and lipids, and many clinical conditions are associated with increased indices of oxidative stress.

Mechanisms for mediating oxidative stress have included antioxidant enzymes and plasma antioxidants many of which are supplanted or, formed via dietary antioxidants. However, when the natural antioxidant system is overwhelmed, the unchecked oxidative stress factors may initiate and propagate biochemical cascades involved in the pathogenesis of many disease states that can be treated by pharmaceutical intervention.

“Elevated oxidative stress” refers to the condition of oxidative stress associated with an increase in oxidative stress as determined by the elevation of at least one biomarker for oxidative stress. The level of elevation necessary to be considered “elevated” depends on the biomarker and depends upon a baseline determined by the level of the biomarker in a population of generally healthy subjects. For example, a measurement of the level of a biomarker one standard deviation above the value determined a healthy population is considered to reflect an elevation of the biomarker that requires clinical assessment and intervention. Thus, one measurement of elevated oxidative stress is a determination that one of the biomarkers for oxidative stress is higher than the value determined in normal populations, using statistical principles. In one recent example, it was found that the future occurrence of cardiovascular events could be correlated with the quintile of the level of C-reactive protein that was present in women that were apparently healthy years earlier. Ridker, 2003. However, when more than one biomarker is evaluated by the clinician, the judgment of the clinician may reflect that a patient has elevated oxidative stress even when none of the measured biomarkers are elevated beyond one standard deviation. This is especially applicable when the patient's history reflects conditions that are known to cause oxidative stress including, for example, patients who smoke, obese patients and diabetic patients.

As used herein a “risk factor” generally refers to one of the well-accepted predictors of adverse cardiovascular events. When a risk factor is present in a large number of human subjects, that group will suffer more cardiovascular events than will a group that does not have the risk factor. Many risk factors have been validated for adverse cardiovascular events by large scale epidemiologic studies such as the Framingham Study. Well-accepted risk factors include, for example, hypertension, elevated blood lipids (hyperlipidemia), diabetes, smoking, and obesity.

A “surrogate” refers to one of the currently recognized predictors of adverse clinical outcome and can be used to estimate risk of the outcome and the response to treatment intended to reduce the risk of the outcome. For adverse cardiovascular events surrogates can include, for example, hypertension and elevated blood lipids. With respect to adverse cardiovascular events, some of the surrogates are also risk factors.

“Biomarkers” report the activity of a biological process of interest and generally include a relevant anatomic, chemical or physiological state that can be measured. For oxidative stress, the biomarkers reflect the intensity of oxidative stress upon the vasculature. While oxidative stress may predispose an individual to adverse cardiovascular events (like the risk factors), the biomarkers for oxidative stress have not been generally accepted as reliable or validated surrogates. Thus, to date, it has not been shown that therapeutically modifying a biomarker for oxidative stress will reduce the number of adverse cardiovascular events in a large population where the biomarker is elevated.

Several biomarkers for oxidative stress are are known and include, for example, C-reactive protein (CRP), fibrinogen, interleukin 6 (IL-6), plasminogin activator inhibitor type 1 (PAI-1), and urinary isoprostanes (8-epi-PGF_{2α} or 8-iso-PGF_{2α}, also known as F₂-isoprostanes). Additional biomarkers are continuing to be identified and should not be limited to those set forth herein. In patients suffering from oxidative stress, these biomarkers may be elevated before any of the traditional risk factors for cardiovascular disease warn of the development of such disease.

Measuring the level of at least one biomarker for oxidative stress refers to a method of determining the concentration of one or more of the biomarkers in a biological sample from the patient. A biological sample may be blood or its components, urine, saliva, tears, tissue, feces and the like that are available from the patient. Commercially available in vitro diagnostic test methods are available for most of the presently known biomarkers and the level of these biomarkers can be determined using the methods described in the instructions from the manufacturers. For example, diagnostic assays for CRP and fibrinogen are available from Dade Behring, Inc., (Deerfield, Ill.); urinary isoprostanes, Oxford Biosciences (Oxford, Mich.); IL-6, Diagnostic Products Corporation (Los Angeles, Calif.); and PAI-1, DakoCytomation (Carpinteria, Calif.).

The terms “treating” and “treatment” as used herein refer to reducing the risk of or preventing an adverse cardiovascular event or reducing oxidative stress in a subject

A “patient at risk” refers to a patient with an increased risk of incurring one or more cardiovascular events. Such risk may be due to disorders, diseases, genetic factors, behaviors, diets, or other conditions or factors. The conditions or factors that frequently lead to elevated cardiovascular risk include, without limitation: current or prior cigarette smoking, diabetes, hemodialysis, receiving an organ transplant, manifest coronary artery disease, history of myocardial infarction, history of transient ischemic attacks or stroke, history of peripheral vascular disease, angina, hypertension, hypercholesterolemia, hyperhomocysteinemia, obesity, atherosclerosis, kidney disease, Chlamydia infection, Bartonella infection, lupus erythematosus and obstructive pulmonary disease.

The term “adverse cardiovascular event,” or simply “cardiovascular event,” as used herein refers, generally, to a disorder or disease of the cardiovascular system resulting from progressive vascular damage. Although the event may have a rather sudden onset, it can also refer to a progressive worsening of such a disorder or disease. Examples of cardiovascular events include, without limitation: claudication, cardiac arrest, myocardial infarction, ischemia, stroke, transient ischemic attacks, worsening of angina, congestive heart failure, or left ventricular hypertrophy. Examples of progressive vascular diseases are are those that affect the cerebral, coronary, renal, or peripheral circulations.

Obesity, is generally defined by a body mass index (BMI) of greater than 30. However, for the purposes herein, obese patients include those patients that are overweight, i.e. those with a BMI of 25 or greater. BMI is calculated by multiplying a patient's weight in pounds by 705 and dividing by the patient's height in inches squared. See, Obesity Education Initiative: Clinical Guidelines on the Identification, Evaluation and Treatment of Overweight and Obesity in Adults, National Institutes of Health, National Heart, Lung and Blood Institute, June 1998.

The “renin-angiotensin-aldosterone system,” or “RAAS,” refers to a biochemical pathway that plays a major role in regulating blood pressure. Renin, an enzyme synthesized, stored, and secreted by the kidneys, potently increases blood pressure. Normally, renin secretion increases when blood pressure is low and decreases when blood pressure is high. Renin functions by acting on angiotensinogen to form the decapeptide angiotensin I. Angiotensin I is rapidly converted to the octapeptide angiotensin II by angiotensin converting enzyme (ACE). Angiotensin II acts by numerous mechanisms to raise blood pressure, including raising total peripheral resistance (in part by constricting precapillary arterioles and, to a lesser extent, postcapillary venules; by enhancing peripheral noradrenergic neurotransmission; and by central nervous system effects), reducing sodium excretion while increasing potassium excretion by the kidneys, and increasing aldosterone secretion by the adrenal cortex (aldosterone acts to retain sodium and to excrete potassium and hydrogen ions). Angiotensin II and aldosterone are also believed to contribute to pathological structural changes in the cardiovascular system, including cardiac hypertrophy (excessive tissue mass), cardiac fibrosis (associated with congestive heart failure and myocardial infarction), and thickening of the intimal surface of blood vessel walls (associated with atherosclerosis).

The term “inhibitor of the renin-angiotensin-aldosterone system” as used herein refers to one or more pharmacologically active, pharmaceutically acceptable agents that inhibit, directly or indirectly, the adverse effects of angiotensin, particularly angiotensin II. Included, without limitation, are agents that: inhibit angiotensin H synthesis; inhibit angiotensin II binding to its receptor; or inhibit renin activity or aldosterone activity.

“Administering” or “administration” refers to providing a patient with a pharmaceutical composition either in one dose or several doses over a course of time up to a period of the remainder of the patient's lifetime. Dosage form, frequency and potency should be therapeutically effective.

An “effective amount” or, as used synonymously, “therapeutically effective amount” of a drug or pharmacologically active agent means a nontoxic but sufficient amount of the drug or agent to provide the desired effect. The amount that is “effective” will vary from subject to subject, depending on the age and general condition of the individual, the particular active agent or agents, and the like, or as determined by one or more attending physicians.

The term “pharmaceutically acceptable,” such as in the recitation of a “pharmaceutically acceptable carrier,” or a “pharmaceutically acceptable salt” means one or more materials that, alone or in combination with one or more other agents and/or excipients, may be administered to a patient without causing significant undesirable biological effects or interacting in a deleterious manner with any of the other components of the composition in which it is contained.

“Pharmacologically active” (or simply “active”), as in “pharmacologically active agent(s), derivative(s) or metabolite(s),” refers to agent(s), derivative(s) or metabolite(s) having the intended pharmacological activity either as administered or from the parent compound. The compounds, i.e., drugs, that are useful in the invention may be in a variety of forms. More specifically, the compounds may be in the form of salts, prodrugs, solvates, hydrates, complexes and polymorphs or combinations thereof, including enantiomers and diastereomers thereof. Those skilled in the art will recognize that salts, e.g., acid or base addition salts, and prodrugs, e.g., esters, are often the form of the active compound found to be most convenient for delivery to a patient in a tablet, capsule or other dosage form. Similarly, those skilled in the art will appreciate that solvates and hydrates of active pharmaceutical compounds are the form of the compound that is often conveniently isolated after manufacturing. Those skilled in the art of organic synthesis are familiar with methodologies to prepare alternative salts, prodrugs, and solvates of the drugs described herein. Thus, while a compound referred to herein may only be identified by a generic name or a tradename, the invention contemplates all forms of the active pharmaceutical agent. The invention is not limited to any of the specific forms delineated above or otherwise.

“Carrier(s),” “diluent(s),” “excipient(s)” and “vehicles” as used herein refer to conventional pharmaceutically acceptable materials used in formulating one or more active ingredients into a final dosage form suitable for drug administration, and include any such materials known in the art that are nontoxic and do not materially interact with other components of a pharmaceutical composition or drug delivery system in a deleterious manner.

An “ACE inhibitor” is an inhibitor of the RAAS and is active by inhibiting the conversion of angiotensin I to angiotensin II by the angiotensin converting enzyme. Most of these compounds can be classified into three groups based on their chemical structure: (1) sulfhydryl—(also called mercapto—) containing ACE inhibitors, including, for example, captopril and agents that are structurally related to captopril, such as fentiapril, pivalopril, zofenopril and alacepril; (2) dicarboxyl-containing ACE inhibitors, including, for example, enalapril and agents that are structurally related to enalapril, such as lisinopril, benazepril, quinapril, moexipril, ramipril, spirapril, perindopril, indolapril, pentopril, indalapril, imidapril and cilazapril; and (3) phosphorus-containing ACE inhibitors, structurally related to fosinopril.

ACE inhibitors are well known in the art, and the use of one or more of any pharmaceutically acceptable ACE inhibitors, including, for example, any of those mentioned in the preceding paragraph and their pharmaceutically acceptable salts, solvates, hydrates, complexes (and combinations thereof), and biologically active, non-toxic enantiomers or diastereomers may be used for carrying out the present invention. Some further examples of ACE inhibitors that may be used in the practice of this invention are, without limitation, AB-103, ancovenin, benazeprilat, BRL-36378, BW-A575C, CGS13928C, CL242817, CV-5975, EU-4865, EU-4867, EU-5476, foroxymithine, FPL 66564, FR-900456, Hoe-065, 15B2, ketomethylureas, KRI-1177, KRI-1230, L681176, libenzapril, MDL-27088, MDL-27467A, moveltipril, MS-41, nicotianamine, phenacein, pivopril, rentiapril, RG-5975, RG-6134, RG-6207, RGH0399, ROO-911, RS-10085-197, RS-2039, RS 5139, RS-86127, RU-44403, S-8308, SA-291, spiraprilat, SQ26900, SQ-28084, SQ-28370, SQ-28940, SQ-31440, utibapril, WF-10129, Wy-44221, Wy-44655, Y23785, P-0154, zabicipril, Asahi Brewery AB-47, alatriopril, BMS 182657, Asahi Chemical C-111, Asahi Chemical C-112, Dainippon DU-1777, mixanpril, zofenoprilat, 1 (-(I-carboxy-6-(4-piperidinyl)hexyl)amino)-1-oxopropyl octahydro-IH-indole-2-carboxylic acid, Bioproject BP1.137, Chiesi CHF 1514, Fisons FPL-66564, idrapril, perindoprilat, Servier S-5590, alacepril, cilazapril, delapril, enalapril, enalaprilat, fosinoprilat, imidapril, ramiprilat, saralasin acetate, temocapril, trandolapril, trandolaprilat, ceranapril, quinaprilat, and those listed in U.S. Pat. No. 6,248,729 which is incorporated herein by reference in its entirety.

Angiotensin II receptor antagonists (also known as angiotensin II antagonists or angiotensin receptor blockers) bind to angiotensin subtype 1 (AT₁) and subtype 2 (AT₂) receptors, as well as to several other receptors. All the known physiological effects of angiotensin II are apparently due to its binding to, and activation of, the AT₁ receptor, which is abundantly expressed in the tissues affected by angiotensin II. AT₂ receptor is common in some fetal tissues but is scarce in adult tissues. Many orally active, nonpeptide angiotensin II receptor antagonists have been developed. Most of these are directed at the AT₁ receptor, but due to concerns about unbalanced activation of the AT₂ receptor, some newer angiotensin II receptor antagonists target both AT₁ and AT₂ receptors. Angiotensin II receptor antagonists are generally highly specific, having very little effect on other hormone receptors or ion channels.

Any active antagonist(s) of the AT₁ angiotensin II receptor may be used in this invention. Some examples of angiotensin II receptor antagonists suitable for use herein are saralasin (including saralasin acetate), candesartan (including candesartan cilexetil), CGP-63170, EMD-66397, KT3-671, LRB/081, valsartan, A-81282, BIBR-363, BIBS-222, BMS-184698, CV11194, EXP-3174, KW-3433, L-161177, L-162154, LR-B/057, LY-235656, PD150304, U-96849, U-97018, UP-275-22, WAY-126227, WK-1492.2K, YM-31472, losartan (including losartan potassium), E-4177, EMD-73495, eprosartan, HN-65021, irbesartan, L-159282, ME-3221, SL-91.0102, tasosartan, telmisartan, UP-269-6, YM-358, CGP-49870, GA-0056, L-159689, L-162234, L-162441, L-163007, PD-123177, A81988, BMS-180560, CGP-38560A, CGP-48369, DA-2079, DE-3489, DuP-167, EXP-063, EXP-6155, EXP-6803, EXP-771 1, EXP-9270, FK-739, HR-720, ICI D6888, ICI-D7155, ICI-D8731, isoteoline, KRI-1177, L-158809, L-158978, L-159874, LR B087, LY-285434, LY-302289, LY-315995, RG-13647, RWJ-38970, RWJ-46458, S-8307, S-8308, saprisartan, sarmesin, WK-1360, X-6803, ZD-6888, ZD-7155, ZD-8731, BIBS39, CI-996, DMP-811, DuP-532, EXP-929, L163017, LY-301875, XH-148, XR-510, zolasartan, and PD-123319. In addition to the above-referenced compounds, their pharmaceutically acceptable salts, solvates, hydrates, complexes (and combinations thereof), and biologically active, non-toxic enantiomers or diastereomers may be used for carrying out the present invention.

Renin inhibitors are compounds that inhibit renin activity such as renin antibodies, analogs of the prosegment of renin, analogs of pepstatin, and analogs of the renin substrate angiotensinogen. As most of these compounds are peptides, they tend to have low oral bioavailability. Various known renin inhibitors are remikiren (Ro 42-5892), A-72517, and A-74273. These compounds are presumed to be active by blocking the stimulation of ACE by renin. In addition to these compounds, their pharmaceutically acceptable salts, solvates, hydrates, complexes (and combinations thereof), and biologically active, non-toxic enantiomers or diastereomers may be used for carrying out the present invention.

Many aldosterone blocking drugs and their effects in humans are known including spironolactones and eplerenones. These drugs are active at the mineralocorticoid receptor level by competitively inhibiting aldosterone binding. In addition, spironolactone has been used for blocking aldosterone-dependent sodium transport in the distal tubule of the kidney in order to reduce edema and to treat essential hypertension and primary hyperaldosteronism. Mantero F et al. (1973). In addition to these compounds, their pharmaceutically acceptable salts, solvates, hydrates, complexes (and combinations thereof), and biologically active, non-toxic enantiomers or diastereomers may be used for carrying out the present invention.

A “statin” is a member of a class of compounds known as a HMG CoA reductase inhibitors. These compounds are frequently prescribed to patients suffering from hyperlipidemia. The members of this class of compounds inhibit 3-hydroxy-3-methylglutaryl coenzyme A (HMG CoA) reductase. This enzyme catalyzes the conversion of HMG CoA to mevalonate, which is an early and rate-limiting step in the biosynthesis of cholesterol. Examples statins that may be used in the invention include but are not limited to lovastatin (see U.S. Pat. No. 4,231,938), simvastatin (see U.S. Pat. No. 4,444,784), pravastatin (see U.S. Pat. No. 4,346,227), fluvastatin (see U.S. Pat. Nos. 5,354,772 and 4,739,073), atorvastatin (see U.S. Pat. No. 5,273,995) atorvastatin calcium (see U.S. Pat. No. 5,273,995), cerivastatin (also called rivastatin; see U.S. Pat. Nos. 5,177,080 and 5,502,199), mevastatin (see U.S. Pat. No. 3,883,140), fluindostatin (Sandoz XU-62-320), velostatin (also called synvinolin; see U.S. Pat. Nos. 4,448,784 and 4,450,171), compactin (see U.S. Pat. No. 4,804,770), dihyrocompactin (see U.S. Pat. No. 4,450,171), dalvastatin (See EP-A 738510) and compounds related to these as described in the cited references, each of which is incorporated by reference herein in its entirety. In addition to these compounds, their pharmaceutically acceptable salts, solvates, hydrates, complexes (and combinations thereof), and biologically active, non-toxic enantiomers or diastereomers may be used for carrying out the present invention.

In one aspect, the present invention relates to a method of treating a mammal, particularly a human, having oxidative stress. By treating the patient for oxidative stress, it is possible to reduce the patient's risk of an adverse event, especially an adverse cardiovascular event. Treatment includes administering an effective amount of one or more pharmaceutically active agents, optionally contained in one or more pharmaceutical composition(s), that reduces the level of oxidative stress in the patient. Such compositions include statins and inhibitors of the RAAS such as ACE inhibitors, angiotensin receptor blockers, renin inhibitors and aldosterone inhibitors. While some of these compositions have previously been described for treating pathologies associated with an adverse cardiovascular event, the present invention provides for treatment for elevated oxidative stress prior to the time when treatment with these composition is otherwise indicated, such as when a patient is suffering from high blood pressure or hyperlipidemia. As described herein, reference to treatment including the administration of a single pharmaceutically active agent or pharmaceutical composition(s) should be interpreted to include the administration of a combination of two or more of the compositions.

In accordance with the present invention, treatment of oxidative stress should begin prior to treatment involving the traditional surrogates for adverse cardiovascular events. Treatment for oxidative stress according to the invention involves the administration of a pharmaceutical composition(s) for treating oxidative stress prior to the time when the administration of such pharmaceutical composition(s) may be otherwise indicated by the recognition of a risk factor or one of the traditional surrogates for an adverse cardiovascular event. For example, in accordance with the present invention, a statin compound should be administered to lower of the level of oxidative stress before a patient exhibits a level of hyperlipidemia that would traditionally suggest treatment with a statin compound is warranted.

The pharmaceutical compositions of the present invention should be administered to a patient having oxidative stress as determined by measuring the level of at least one biomarker for oxidative stress in the patient. Administration of the pharmaceutical composition(s) to treat the oxidative stress can begin based upon a finding of elevated oxidative stress. Such treatment is particularly appropriate in the absence of an elevated level of the traditional risk factors for cardiovascular disease in a patient such as hypertension, total cholesterol and LDL. In addition, when a patient has other risk factors suggesting a future cardiac event, such as, for example, the patient is a smoker, is obese, or has diabetes, treatment can be initiated when, in the discretion of the treating physician, the level of one or more of the biomarkers is greater than that of the normal population but is not elevated as specifically defined herein. Example 1 below provides an example of a method for determining when treatment for oxidative stress is appropriate. This example is not intended to limit the invention in any way as a physician or other professional who understands the risks associated with the administration of the pharmaceutical compositions identified herein can decide with the patient's input when it is appropriate to begin treatment with one or more pharmaceutically active agents or pharmaceutical compositions for lowering at least one biomarker for oxidative stress.

In another aspect, the invention is directed to a method of treating a mammalian subject, particularly a human, by measuring in the subject the level of one or more biomarker(s) for oxidative stress and relating the level of the biomarker to the risk of an adverse event, especially a cardiovascular event. When such oxidative stress is determined to be elevated, the attending physician may exercise his/her discretion to administer to the patient one or more pharmaceutical agents and/or pharmaceutical compositions for treating such oxidative stress. Thus, in one aspect, the method of lowering the risk of an adverse event, especially an adverse cardiovascular event, in human subjects includes measuring in the subject the level of at least one biomarker for oxidative stress and treating those subjects having elevated oxidative stress to lower the level or, in the least, maintaining the level of the at least one biomarker.

In another aspect, the invention is related to reducing the risk of an adverse event, especially an adverse cardiovascular event, by preventing an increase in the level of oxidative stress in a patient. Accordingly, by preventing an increase in the level of oxidative stress, the patient is protected from the progressive damage resulting from unchecked escalation of oxidative stress. Thus, while a patient's lifestyle or other factors that are associated with increasing oxidative stress may prevent the patient from responding to treatment so that a patient's level of oxidative stress is lowered, the risk of a adverse event, especially a cardiovascular event, can be reduced in those patients by treatment, according to the methods of the present invention, that prevents an increase in oxidative stress. Alternatively, the risk for an adverse event such as an adverse cardiovascular event may be lowered by measuring the oxidative stress of a subject by testing for at least one biomarker for oxidative stress, determining whether the at least one tested biomarker is indicative of oxidative stress, and if the at least one tested biomarker is indicative of oxidative stress, the subject is then treated to reduce or prevent an increase of the level of the least one biomarker for oxidative stress.

In another aspect, the invention is directed to a method for ascertaining whether a subject has oxidative stress. The method includes measuring the oxidative stress of a subject by testing for at least one biomarker for oxidative stress from said subject and determining whether the at least one tested biomarker is indicative of oxidative stress. If the at least one tested biomarker is indicative of oxidative stress, the subject would be treated to reduce or prevent an increase of the level of the least one biomarker for oxidative stress. Because oxidative stress has been implicated in an adverse risk of disease, particular adverse cardiovascular events, ascertaining for oxidative stress in subjects and treating such subjects would be beneficial in reducing or preventing adverse risks.

In another aspect, the present invention is related to a method of determining the effectiveness of a treatment for the reducing the risk of an adverse event in a human subject comprising monitoring, during treatment of the subject, the risk of an adverse event by measuring the change in the level of oxidative stress in a patient during the treatment. For example, the level of one or more biomarkers for oxidative stress can be measured during the administration of a pharmaceutical composition(s) for treating oxidative stress. The effectiveness of the treatment can be determined by examining whether the level of the one or more biomarkers for oxidative stress changes during the administration of the composition for reducing the oxidative stress. In an additional aspect of the present application, an algorithm as exemplified in Examples 1 and 2, below, can be used to determine the effectiveness of treatment. Those skilled in the art of pharmaceutical administration will able to readily design a similar algorithm to determine effectiveness of a treatment for lowering oxidative stress.

Similarly, the present invention provides in one aspect a method of determining when to adjust a treatment for reducing the risk an adverse event, especially an adverse cardiovascular event. When the treatment includes the administration of a pharmaceutical composition(s) for treating oxidative stress according to the present invention, the patient's risk of an adverse event can be determined by measuring the level of oxidative stress in the patient. The administration of the pharmaceutical composition(s) of the present invention can be adjusted based upon the calculated risk. If the risk has been lowered since the patient began treatment, the dosage of the composition may be adjusted by termination of the dose or modification of the amount of the dosage of such pharmaceutical composition(s). It is in the discretion of the physician and the patient to determine when the risk of the event is significant enough to continue the administration. For example, when the risk, cost or inconvenience of the administration outweigh the risk of the event, the dosage may be lowered or discontinued at the discretion of the physician or the patient. Additionally, the patient may adjust his/her lifestyle, such as losing weight or quitting smoking. These factors may also be considered as part of the cost/benefit analysis of continuing treatment. At the same time, those patients who cannot or will not adjust their lifestyles to lower their risk may decide that the continuation of the treatment is appropriate to maintain a lower level of risk than observed without the treatment.

Accordingly, in one aspect, the present invention is directed to a method of treating a patient at risk of an adverse cardiovascular event that includes determining the patient's risk of an adverse cardiovascular event by measuring the patient's level of oxidative stress. When a patient has elevated oxidative stress, treatment can include administering to the patient an amount of one or more pharmaceutical agents and/or pharmaceutical compositions according to the present invention for treating oxidative stress. During administration, the level of oxidative stress can be measured and treatment decisions can be made based upon the increase or decrease of oxidative stress. Such decisions include terminating the treatment, or modifying the dosage strength of such pharmaceutical agent(s) and/or of the pharmaceutical composition(s) when it is concluded that the consequences of further administration at the currently prescribed dosage strength outweighs a benefit of a lower risk.

In yet another aspect, the invention is directed to a method for lowering the risk of an adverse cardiovascular event in a subject having elevated oxidative stress. The method includes: measuring the oxidative stress of a subject by testing for at least one biomarker for oxidative stress from said subject; determining whether the at least one tested biomarker is indicative of oxidative stress; and wherein when the at least one tested biomarker is indicative of oxidative stress, treating said subject to reduce oxidative stress. Any one or more biomarkers for oxidative stress may be used for determining whether the subject's level of oxidative stress. Preferably, at least two biomarkers are used for the determination. The step of determining includes: (a) assigning previously measured biomarker data into a number of windows, wherein each window has a weighted value; (b) comparing the value of the tested biomarker to at least one window so as to obtain an oxidative stress score component; (c) aggregating the oxidative stress score component to produce an oxidative stress score; and (d) comparing the oxidative stress score to a previously determined threshold value to determine whether treatment is needed. The phrase “previously measured biomarker data” means previously reported data obtained from any source, including published literature. Generally, such biomarker data has been reported in the medical literature and is generally presented as a mean plus or minus a standard deviation or as a distribution that is scaled as a tertile, quartile or quintile. See, for instance, Example 2.

The previously measured biomarker data is divided up into a series of windows having assigned weighted values representing the positive or negative contribution to risk by the quantitative standard deviation from a normal population value. The number of windows depends on how the data is reported. For instance, if the previously measured biomarker data is reported as a quintile, then the data may distributed over a series of five windows, each window have an assigned weighted value. For instance, the first (highest) quintile could be assigned to the first window, the second (second highest) quintile could be assigned to a second window, the third (middle) quintile could be assigned to the third window, the fourth (second lowest) quintile could be assigned to the fourth window, and the fifth (lowest) quintile would be assigned to the fifth window. The first, second, third, fourth, and fifth windows could have an assigned weighted value of +5, +3, +1, −1 and −3, respectively. If the previously measured biomarker data is reported as a quartile, then the data may be distributed over a series of four windows, each window having an assigned weighted value. The first, second, third, and fourth window would have an assigned weighted value of +4, 2, 0, and −2, respectively. If the previously measured biomarker data is reported as a tertile, then the data may be distributed over a series of three windows, each window having an assigned weighted value. The first, second and third windows would have an assigned weighted value of +4, +1, and −2.

In some instances, previously determined biomarker data is reported as a mean plus or minus a standard deviation. The data may be distributed over a series of windows, each window having an assigned weighted value. For instance, the data may be distributed over a series of five windows wherein the first window represents an area greater than two standard deviations above the mean, the second window represents an area between greater than 1.6 standard deviations above the mean and two standard deviations above the mean, the third window represents an area between 1.6 standard deviation above the mean and one standard deviation above the mean; the fourth window represents one standard deviation above and below the mean; and the fifth window represents the area of more than one standard deviation below the mean. The assigned weighted values for the first to fifth windows could have a value of +5, +3, +1, −1, and −3, respectively.

The conversion of the previously reported biomarker data into a series of windows having assigned values can be performed by the physician. Alternatively, prior arrangements of such windows may be simply provided to the physician in any suitable form such as a table or graph. The physician who would then compare the value of the patient's tested biomarker to at least one window to obtain an oxidative stress score component.

Once the previously determined biomarker data has been distributed into various windows, the value of the subject's tested biomarker is then compared to at least one window so as to obtain an oxidative stress score component. For instance, if the subject's tested biomarker falls into the second (second highest) quintile for a previously determined biomarker data that has been scaled as a quintile, then the subject's oxidative score component is 3. The oxidative stress score components of all of the tested biomarkers are then aggregated to produce an oxidative stress score. The step of aggregating includes summing the oxidative stress score component of each tested biomarker and dividing the sum by a total number of tested biomarkers. The subject's oxidative stress score is then compared to a previously determined threshold value to determine whether treatment is needed to reduce oxidative stress. The phrase “predetermined threshold value” refers to a value of an oxidative stress score that is approximately one standard deviation greater than the value of an oxidative stress score found for a normal population. Thus, patients having an oxidative stress score of about 3.0 or higher, usually about 3.25 or higher should be treated for lowering at least one of the biomarkers for oxidative stress. If a patient has at least one risk factor for a cardiovascular event, a physician may want to treat patients having an oxidative stress score of about 2.0 or higher, usually about 2.5 or higher. The upper limits for the oxidative stress scores depend, in part, on the manner in which the previously reported biomarker data are distributed. For instance, if the biomarker data is scaled as a quintile or a mean plus or minus a standard deviation, then the maximum oxidative stress score component will be 5. For quartiles and tertiles, the maximum oxidative stress score component will be 4. For normal persons without cardiovascular disease, an oxidative stress score of zero or less would be expected. Non-limiting risk factors for a cardiovascular event include, without limitation, hypertension, hypercholesterolemia, hyperhomocysteinemia, obesity, diabetes mellitus, or smoking. Generally, if the subject's oxidative stress score is above the previously determined threshold value, this indicates a risk of a cardiovascular event. If the subject's oxidative stress score is below said threshold value, the subject has a lower risk of an oxidative stress-related event relative to the normal population which is defined herein as a population which does not exhibit evidence of cardiovascular disease.

In yet another aspect, the invention is directed to a method for evaluating the level of oxidative stress in a subject. The method includes measuring the oxidative stress of a subject by testing for at least one biomarker for oxidative stress from said subject; assigning previously measured biomarker data into a number of windows, wherein each window has a weighted value; comparing the value of the tested biomarker to at least one window so as to obtain an oxidative stress score component; aggregating the oxidative stress score component to produce an oxidative stress score; and comparing the oxidative stress score to a previously determined threshold value. The measurement of oxidative stress in a subject is valuable in that oxidative stress has been implicated in a variety of conditions, including inflammation.

In yet another aspect, the invention is directed to a method for determining the effectiveness of at one pharmaceutical composition for reducing the risk of an adverse cardiovascular event in a subject having elevated oxidative stress. The method includes measuring a first level of oxidative stress of a subject by testing for at least one biomarker for oxidative stress, administering at least one pharmaceutical composition to said subject to reduce the level of at least one biomarker for oxidative stress, measuring a second level of oxidative stress of said subject by testing for the at least one biomarker for oxidative stress; and comparing the values of the first and second levels of oxidative stress so as to determine whether the at least one pharmaceutical composition is effective in reducing the risk of an adverse cardiovascular event. Thus, by measuring and comparing the levels of oxidative stress of a subject before and during treatment of a subject with the pharmaceutical composition, it is possible to determine the effectiveness of the treatment in reducing the level of oxidative stress and risk of an adverse cardiovascular event. For instance, if second level of oxidative stress found to less than the value of the first level of oxidative stress and a predetermined threshold value, then the at least one pharmaceutical composition is effective in reducing the risk of an adverse cardiovascular event. However, if the value of the second level of oxidative stress is equal to and greater than the value of the first level of oxidative stress, then the at least one pharmaceutical composition or dosage amount of the least one pharmaceutical composition is ineffective or insufficient in reducing the risk of an adverse cardiovascular event.

If the treatment is effective such as in a case where if step (d) value of the second level of oxidative stress is less than the value of the first level of oxidative stress and a predetermined threshold value, then the method further includes a step (e) terminating or reducing the amount of the at least one pharmaceutical composition being administered to said subject. The amount of dosage reduction for each particular patient will depend on a variety of factors, including age, body weight, general health, gender, diet, time of administration and so forth. Generally, the amount of dosage reduction may range from about 5% to 75% of the original dosage amount.

If the treatment is ineffective or insufficient such in a case where if step (d) value of the second level of oxidative stress is equal to or greater than the value of the first level of oxidative stress, then the method further comprises a step(e) terminating the administration of the at least one pharmaceutical composition to said subject and choosing another pharmaceutical composition to administer. Then the monitoring of oxidative stress level is repeated as above with the new regimen.

In another aspect, the invention is directed to a method for evaluating the level of oxidative stress in a subject by measuring the oxidative stress of a subject by testing for at least one biomarker for oxidative stress from said subject. The value of the tested biomarker would then be compared with at least one window of an arrangement of windows as discussed above so as to obtain an oxidative stress score component. The component is then used to determine an overall oxidative stress score by the aggregating process described above and the resultant oxidative stress score is then compared to a previously determined threshold value as discussed above.

In order to treat patients to reduce the risk of a cardiovascular event, the pharmaceutical agents and pharmaceutical compositions of the present invention should be administered in a therapeutically effective amount. The amount of active ingredient that may be combined with the, for example, carriers, diluents or excipients to produce a single dosage form will vary depending upon the host treated and the particular mode of administration. Alternatively, dosage forms for many of the pharmaceutical compositions used to carry out the methods of the present invention are known in the art. The daily dose is usually administered in one to four doses per day. It will be understood, however, that the specific dose level for any particular patient will depend upon a variety of factors including, for example, the activity of the specific compound employed, the age, body weight, general health, gender, diet, time of administration, route of administration, and rate of excretion, and drug combinations.

Many of the pharmaceutical compositions identified herein are currently being prescribed for treatment of various conditions associated with cardiovascular disease and other disorders based on traditional risk factors, conditions, or identified cardiovascular disease states. Thus, safety considerations associated with these compositions are applicable to the treatment of oxidative stress. The presently recommended dosages for these compositions can be used as a starting point as a dosage strength for treating oxidative stress. The dosage may be adjusted based upon the change in the level of oxidative stress during administration of the at least one composition. In one aspect of the present invention, the dosage strength for treating oxidative stress is lower than the recommended dosage of the composition when used for treating the disorder that such composition is traditionally used to treat. Because the present invention is directed to an early intervention therapy, or preventative therapy, the dosage need only be sufficient to lower, or prevent an increase of, the level of oxidative stress without having to treat, for example, hyperlipidemia or hypertension. This may be a lower dose than that currently recommended.

Many statin compositions are currently available by prescription. The recommended dose for these compositions include a range of between 5 and 80 mg per day for treating hyperlipidemia, depending on the factors described above. Information regarding the recommended dosages for statin compounds is readily available in the Physicians' Desk Reference as updated annually or from the manufacturers. For example, the following daily dosages are recommended: atorvastatin (LIPITOR®), 10-80 mg; fluvastatin (LESCOL®), 20-80; lovastatin (MEVACOR®), 20-80; pravastatin (PRAVACHOL®), 10-40 mg; and simvastatin (ZOCOR®), 10-80 mg.

ACE Inhibitors are generally prescribed to treat hypertension at between 1-40 mg per day depending on the composition and patient considerations. Many ACE inhibitors are available by prescription and are described in the Physicians' Desk Reference as updated annually. Information is also available from the manufacturers. Examples of recommended daily doses for these compounds are as follows: quinapril (ACCUPRIL®), 5-40 mg; ramipril (ALTACE®), 1.25-10 mg; captopril (CAPOTEN®), 12.5-50 mg; perindopril (ACEON®), 2-8 mg; benazepril (LOTENSIN®), 5-40 mg; cilazapril (VASCACE®, INIBACE®), 1-10 mg; lisinopril (ZESTORETIC®, ZESTRIL®, ), 5-40 mg; fosinopril (MONOPRIL®, DYNACIL®, STARIL®), 10-40 mg; and enalapril (VASOTEC®), 5-40 mg.

Many angiotensin receptor blockers are available by prescription and are described in the Physicians' Desk Reference as updated annually. Information is also available from the manufacturers. Examples and recommended daily dosages of these compositions include: candesartan (ATACAND®), 4-16 mg; eprosartan (TEVETEN®), 300-800 mg; irbesartan (AVAPRO®), 75-300 mg; losartan (COZAAR®), 25-100 mg; telmisartan (MICARDIS®), 20-80mg; valsartan (DIOVAN®), 40-160 mg.

Aldosterone inhibitor eplerenone is generally administered in the range of 50-200 mg per day to treat hypertension. Another aldosterone inhibitor spironolactone is used in the treatment of other hyperaldosterone-related diseases such as liver cirrhosis and congestive heart failure. Saunders F J et al., 1978. In addition, spironolactone at a dosage ranging from 25-100 mg daily is used to treat diuretic-induced hypokalemia, when orally-administered potassium supplements or other potassium-soaring regimens are considered inappropriate.

The pharmaceutical composition described herein and used for the methods of the present invention can be administered orally, topically, parenterally, by inhalation or spray, vaginally or rectally in dosage unit formulations containing conventional non-toxic pharmaceutically acceptable carriers, adjuvants and vehicles and administered with the presently recommended frequency or as necessarily adjusted to provide appropriate total daily doses according to the methods of the present invention. The term parenteral as used herein includes percutaneous, subcutaneous, intravascular (e.g., intravenous), intramuscular, or intrathecal injection or infusion techniques and the like. A solid pharmaceutical composition of the present invention may be blended with at least one pharmaceutically acceptable excipient, diluted by an excipient or enclosed within such a carrier which can be in the form of a capsule, sachet, tablet, buccal, lozenge, paper, or other container. When the excipient serves as a diluent, it may be a solid, semi-solid, or liquid material which acts as a vehicle, carrier, or medium for the active ingredient. Thus, the formulations can be in the form of tablets, pills, powders, elixirs, suspensions, emulsions, solutions, syrups, capsules (such as, for example, soft and hard gelatin capsules), suppositories, sterile injectable solutions, and sterile packaged powders.

In addition to known pharmaceutical compositions, other compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Typically, tablets and capsules contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases, such coatings may be prepared by known techniques to delay disintegration and absorption until the pharmaceutical composition, or part of the composition, reaches the gastrointestinal tract and thereby provide a sustained action over a longer period of time. For example, a time delay material such as glyceryl monosterate or glyceryl distearate may be employed.

Formulations for oral use may also be presented as hard or soft capsules, including gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. The capsules may also be coated for delayed or targeted release. Formulations for oral use may also be presented as lozenges.

Aqueous suspensions contain the active materials in admixture with excipients suitable for the manufacture of such aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions used in the methods of the present invention may also be in the form of various emulsions including, for example, oil-in-water emulsions wherein the oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensati on products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents.

The pharmaceutical compositions used for the present invention may also be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are, for example, water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The pharmaceutical compositions used to carry out the present invention may also be administered in the form of suppositories, e.g., for rectal or vaginal administration of the drug. These compositions can be prepared by mixing the drug with a suitable non-irritating excipient that is solid at ordinary temperatures but liquid at the rectal or vaginal temperature and will therefore melt accordingly to release the drug. Such materials include cocoa butter and polyethylene glycols.

The pharmaceutical compositions may further be prepared to be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The pharmaceutical compositions used for the methods of the present invention can also be administered by a transdermal device. Preferably, topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch.

Any of the active agents may be administered in the form of a salt, ester, amide, prodrug, active metabolite, analog, hydrate, solvate, complexes, and biologically active, non-toxic enantiomers and diastereomers, or combinations thereof, provided that the agent is pharmaceutically acceptable and pharmacologically active in the present context. The active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Hydrates and solvates of the compounds along with polymorphs thereof are also forms of the pharmaceutical agents used in the compositions and methods of the present invention and may be formed according to techniques known to one having ordinary skill in the pharmaceutical arts. Such pharmaceutical agents can also be present in the form of a complex, particularly organo-metallic complexes, as appropriate and as prepared using processes also known by the skilled artisan.

The following are provided for exemplification purposes only and are not intended to limit the scope of the invention described in broad terms above. All references cited in this disclosure are incorporated herein by reference.

### EXAMPLES

### Example 1

The risk of an adverse cardiovascular event can be maintained at low levels, lowered or eliminated by treating patients at risk for such events prior to when it is conventionally considered appropriate to treat based upon the measurement of traditional risk factors for cardiovascular disease such as total cholesterol, LDL cholesterol and hypertension. Treatment decisions can now be based upon the level of at least one biomarker for oxidative stress. Accordingly, the first step in determining whether to treat a patient to lower the risk of an adverse cardiovascular event is to measure at least one biomarker for oxidative stress. Such biomarkers include, for example, CRP, IL-6, , fibrinogen, PAI-1, and urinary isoprostanes. Commercially available in vitro assays are available for each of these biomarkers.

Once the level of one or more biomarkers has been measured, the next step includes placing the patient's scores into a window for each of the measurements. For example, when the biomarker values for subjects at lowest to highest values are express in quintiles, one would assign the following weighted values to each quintile.
Highest quintile=5
Second highest quintile=3
Middle quintile=1
Second lowest quintile=−1
Lowest quintile=−3

Then, the weighted values are added together and divided by the number of biomarkers measured to determine the patient's oxidative score.

In this Example, an oxidative stress score of 3.25 is approximately equal to a value about one standard deviation greater than the normal population. A patient having a score of 3.25 or higher should be treated for lowering at least one of the biomarkers for oxidative stress according to the methods of the present invention. In addition, a physician may treat patients having an oxidative stress score between 2.75 and 3.25 if other risks are present such as obesity, diabetes or smoking. When the patient has a history of a previous cardiovascular event, the physician may consider treating a patient having an oxidative score as low as 2.

The following is an example how an oxidative score can be determined. First, the patient's measurement for oxidative stress are placed in the appropriate quintile.

| | Quintile | | | | |
|---|---|---|---|---|---|
| | high | second high | middle | second low | low |
| HsCRP | X | | | | |
| IL-6 | | | X | | |
| Urine isoP | | X | | | |
| Fibrinogen | | X | | | |
| PAI-1 | X | | | | |

The oxidative score is determined by assigning a value to each quintile (high=5, second high=3, middle=1, second low=−1, low =−1). The values are added and divided by the number of values: 5+1+3+3+5=17/5=oxidative score=3.4. If, for example, IL-6 and PAI-1 were not measured, the calculation is: 5+3+3=11/3=oxidative score=3.67. In this Example, an oxidative stress score of 3.25 is greater than a value about one standard deviation above the mean for that of the normal population. A patient having a score of 3.25 or higher should be treated for lowering at least one of the biomarkers for oxidative stress according to the methods of the present invention. In addition, a physician may treat patients having an oxidative stress score between 2.75 and 3.25 if other risks are present such as obesity, diabetes or smoking. When the patient has a history of a previous cardiovascular event, the physician may consider treating a patient having an oxidative score as low as 2. It would be apparent to those skilled in the art that any biomarker of oxidative stress could be added to the above algorithm.

### Example 2

In this Example, a patient is evaluated to determine whether a risk of an adverse cardiovascular event exists and whether treatment is warranted. As in Example 1, the first step in determining whether to treat a patient to lower the risk of an adverse cardiovascular event is to measure at least one biomarker for oxidative stress. For this Example, biomarkers CRP is expressed in quintiles, IL-6 in quartiles, fibrinogen and urine isoprostane as deviations from mean, a new biomarker expressed in tertiles is incorporated, and PAI-1 is not measured. Further the results are shown after initiation of treatment with a satisfactory reduction in the oxidative stress score.

| Biomarker | value | score |
|---|---|---|
| CRP | fifth quintile | +5 |
| Fibrinogen | 1.8 SD above mean | +3 |
| IL-6 | fourth quartile | +4 |
| New marker | second tertile | +1 |
| Urine isoprostane | 2.1 SD above mean | +5 |

The sum of oxidative stress components measured is 18; five were measured, and the resultant oxidative stress score is 3.6.

Even though the hypothetical patient has no risk factors and has not suffered a previous cardiovascular event, treatment is instituted with an ACE inhibitor, enalpril (10 mg), and a statin, rosuvastatin (20 mg).

After 3 months of treatment, the biomarker levels are again measured with the following results:

| | | |
|---|---|---|
| CRP | third quintile | +1 |
| Fibrinogen | +1.7 SD | +3 |
| IL-6 | third quartile | +2 |
| New marker | first tertile | −2 |
| Urine isoprostane | +1.8 SD | +3 |

Now the sum of the components is 7, five components were measured, and the oxidative stress score on treatment is 1.4.

Although various specific embodiments of the present invention have been described herein, it is to be understood that the invention is not limited to those precise embodiments and that various changes or modifications can be affected therein by one skilled in the art without departing from the scope and spirit of the invention.

### References

Mantero, F., et al., Clin. Sci. Mol. Med., 45(Suppl 1)219s-224s (1973)

F. J. Saunders et al., Aldactone; Spironolactone: A Comprehensive Review, Searle, New York (1978).

Bermudez, Edmund, et al., Relation Between Markers of Systemic Vascular Inflammation and Smoking in Women, American Journal of Cardiology 89(9):1117-1119 (2002).

Ridker, P., Clinical Application of C-Reactive Protein for Cardiovascular Disease Detection and Prevention, Circulation Vol. 107(3):363-369 (2003).

Dzau, Victor, Tissue Angiotensin and Pathobiology of Vascular Disease: A Unifying Hypothesis, Hypertension, 374(4):1047-1052 (2001).

Law, M. R., et al., Quantifying effect of statins on low density lipoprotein cholesterol, ischameic heart disease, and stroke: systematic review and meta-analysis, BMJ, Vol. 326 (June 2003).

Law, M. R., et al., Value of low dose combination treatment with blood pressure lowering drugs: analysis of 354 randomised trials, BMJ, Vol. 326:1427 (June 2003).

Wald, N. J., et al., A strategy to reduce cardiovascular disease by more than 80%, BMJ, Vol. 326:1419:1423 (June 2003).

Bazzano, Lydia, et al., Relationship Between Cigarette Smoking and Novel Risk Factors for Cardiovascular Disease in the United States, Ann Inter Med 138(11):891-897 (2003).

Pearson, Thomas, et al., Markers of Inflammation and Cardiovascular Disease: Application to Clinical Public Health Practice: A Statement for Healthcare Professionals From the Centers for Disease Control and Prevention and the American Heart Association, Circulation 108(3):499-511 (2003).

Block, G., et al., Factors Associated with Oxidative Stress in Human Populations, American Journal of Epidemiologyl 56 (3):274-285 (2002).

Keaney, John, et al., Obesity and Systemic Oxidative Stress: Clinical Correlates of Oxidative Stress in the Framingham Study, Arterioscler Throm Vasc. Biol. 23:434-439 (2003).

Morrow, J., Is Oxidant Stress a Connection Between Obesity and Atherosclerosis?, Areterioscler Throm Vasc. Biol, 23:368-370 (2003).

Hackham D G, et al., Emerging Risk Factors for Atherosclerotic Vascular Disease: A Critical Review of the Evidence, JAMA 290:932-940 (2003)

Nordt T K, et al., Plasminogen activator inhibitor type-1 (PAI-1) and its role in cardiovascular disease, Thromb Haemostasis 82 Suppl 1:14-8 (1999).

1. A method of treating a subject having oxidative stress, said method comprising administering to said subject an effective amount of at least one pharmaceutical composition for reducing or preventing an increase of the level of at least one biomarker for oxidative stress.

2. The method of claim 1 wherein the at least one pharmaceutical composition comprises an inhibitor of the renin-angiotensin-aldosterone system.

3. The method of claim 2 wherein the inhibitor of the renin-angiotensin-aldosterone system comprises an angiotensin converting enzyme inhibitor.

4. The method of claim 3 wherein the angiotensin converting enzyme inhibitor is selected from the group consisting of AB-103, ancovenin, benazeprilat, BRL-36378, BW-A575C, CGS13928C, CL242817, CV-5975, EU-4865, EU-4867, EU-5476, foroxymithine, FPL 66564, FR-900456, Hoe-065, 15B2, ketomethylureas, KRI-1177, KRI-1230, L681176, libenzapril, MDL-27088, MDL-27467A, moveltipril, MS-41, nicotianamine, phenacein, pivopril, rentiapril, RG-5975, RG-6134, RG-6207, RGH0399, ROO-911, RS-10085-197, RS-2039, RS 5139, RS-86127, RU-44403, S-8308, SA-291, spiraprilat, SQ26900, SQ-28084, SQ-28370, SQ-28940, SQ-31440, utibapril, WF-10129, Wy-44221, Wy-44655, Y23785, P-0154, zabicipril, Asahi Brewery AB-47, alatriopril, BMS 182657, Asahi Chemical C-111, Asahi Chemical C-112, Dainippon DU-1777, mixanpril, zofenoprilat, 1(-(I-carboxy-6-(4-piperidinyl)hexyl)amino)-1-oxopropyl octahydro-IH-indole-2-carboxylic acid, Bioproject BP1.137, Chiesi CHF 1514, Fisons FPL-66564, idrapril, perindoprilat, Servier S-5590, alacepril, cilazapril, delapril, enalapril, enalaprilat, fosinoprilat, imidapril, ramiprilat, saralasin acetate, temocapril, trandolapril, trandolaprilat, ceranapril, and uinaprilat.

5. The method of claim 2 wherein the inhibitor of the renin-angiotensin-aldosterone system comprises an angiotensin receptor blocker.

6. The method of claim 5 wherein the angiotensin receptor blocker is selected from the group consisting of saralasin, saralasin, candesartan, CGP-63170, EMD-66397, KT3-671, LRB/081, valsartan, A-81282, BIBR-363, BIBS-222, BMS-184698, CV11194, EXP-3174, KW-3433, L-161177, L-162154, LR-B/057, LY-235656, PD150304, U-96849, U-97018, UP-275-22, WAY-126227, WK-1492.2K, YM-31472, losartan, E-4177, EMD-73495, eprosartan, HN-65021, irbesartan, L-159282, ME-3221, SL-91.0102, tasosartan, telmisartan, UP-269-6, YM-358, CGP-49870, GA-0056, L-159689, L-162234, L-162441, L-163007, PD-123177, A81988, BMS-180560, CGP-38560A, CGP-48369, DA-2079, DE-3489, DuP-167, EXP-063, EXP-6155, EXP-6803, EXP-7711, EXP-9270, FK-739, HR-720, ICI D6888, ICI-D7155, ICI-D8731, isoteoline, KRI-1177, L-158809, L-158978, L-159874, LR B087, LY-285434, LY-302289, LY-315995, RG-13647, RWJ-38970, RWJ-46458, S-8307, S-8308, saprisartan, sarmesin, WK-1360, X-6803, ZD-6888, ZD-7155, ZD-8731, BIBS39, CI-996, DMP-811, DuP-532, EXP-929, L163017, LY-301875, XH-148, XR-510, zolasartan, and PD-123319.

7. The method of claim 2 wherein the inhibitor of the renin-angiotensin-aldosterone system comprises a renin inhibitor.

8. The method of claim 7 wherein the renin inhibitor is selected from the group consisting of renin antibodies, analogs of the prosegment of renin, analogs of pepstatin, and analogs of the renin analogs of the prosegment of renin, analogs of pepstatin, and analogs of the renin substrate angiotensinogen, remikiren (Ro 42-5892), A-72517, and A-74273.

9. The method of claim 1 wherein the at least one pharmaceutical composition comprises an inhibitor of the aldosterone system.

10. The method of claim 9 wherein the inhibitor of the aldosterone system is an aldosterone antagonist.

11. The method of claim 10 wherein the inhibitor is selected from the group consisting of spironolactones and eplerenones.

12. The method of claim 1 wherein the at least one pharmaceutical composition comprises a statin compound.

13. The method of claim 12 wherein the statin compound is selected from the group consisting of lovastatin, simvastatin, pravastatin, fluvastatin, atorvastatin, atorvastatin calcium, cerivastatin, mevastatin, fluindostatin, velostatin, compactin, dihydrocompactin, and dalvastatin.

14. The method of claim 1 wherein the at least one biomarker for oxidative stress is at least one member of the group selected CRP, IL-6, PAI-1, fibrinogen and urinary isoprostane.

15. The method of claim 1 wherein said administering occurs prior to treatment of at least one disease state associated with a surrogate for cardiovascular disease.

16. A method of treating a subject at risk of an adverse cardiovascular event as the result of having elevated oxidative stress, said method comprising administering to said subject an effective amount of at least one pharmaceutical composition for reducing or preventing the increase of the level of at least one biomarker for oxidative stress.

17. The method of claim 16 wherein said administering occurs prior to treatment of at least one disease state associated with a surrogate for cardiovascular disease.

18. The method of claim 16 wherein the at least one surrogate for cardiovascular disease is selected from the group consisting of hypertension, hypercholesterolemia, diabetes mellitus, and hyperhomocysteinemia.

19. A method of reducing the risk of an adverse cardiovascular event in a subject having elevated oxidative stress, said method comprising administering to said subject an effective amount of at least one pharmaceutical composition for reducing or preventing the increase of the level of at least one biomarker for oxidative stress.

20. The method of claim 19 wherein said administering occurs prior to treatment of at least one disease state associated with a surrogate for cardiovascular disease.

21. The method of claim 19 wherein the at least one surrogate for cardiovascular disease is selected from the group consisting of hypertension, hypercholesterolemia, diabetes mellitus, and hyperhomocysteinemia.

22. A method for treating a subject suspected of having oxidative stress, said method comprising:
measuring the oxidative stress of a subject by testing for at least one biomarker for oxidative stress from said subject;
determining whether the at least one tested biomarker is indicative of oxidative stress; and
wherein when the at least one tested biomarker is indicative of oxidative stress, treating said subject to reduce or prevent an increase of the level of the least one biomarker for oxidative stress.

23-69. (Cancelled)
